(19) United States
(12) Patent Application Publication
(10) Pub. No.: US 2012/0009591 A1
(43) Pub. Date: Jan. 12, 2012

(54) METHOD FOR DETECTION OF AN ENZYME-SUBSTRATE REACTION

(76) Inventor: Ewa Pol, Uppsala (SE)

(21) Appl. No.: 13/257,760

(22) PCT Filed: Mar. 18, 2010
(86) PCT No.: PCT/SE10/50313

§ 371 Date: Sep. 20, 2011

(30) Foreign Application Priority Data
Mar. 20, 2009 (SE) 0950175-0

(51) Int. Cl. G01N 21/55; C12M 1/40; G01N 33/53

(52) U.S. Cl. 435/7.1; 435/287.2; 435/288.7

(57) ABSTRACT

The present invention relates to a method for detection of an enzyme-substrate reaction by capturing the product of the reaction on a solid support. More closely, the invention relates to a solid phase system for enzyme characterisation and screening of enzyme-targeted drugs. The method comprises the following steps a) rapidly mixing an enzyme solution with a substrate solution to start a reaction between enzyme and substrate wherein said reaction may result in product formation; and b) monitoring any binding of the product to a capturing agent immobilised on a solid surface, wherein said capturing agent is directed against the product of the enzymatic reaction. In a preferred embodiment the method comprises a further step c) measuring the initial rate of the product binding to the capturing agent and thereby characterize the enzymatic reaction. The method may also be used for drug screening.

### CROSS-REFERENCE TO RELATED APPLICATIONS

This application is a filing under 35 U.S.C. §371 and claims priority to international patent application number PCT/SE2010/050313 filed Mar. 18, 2010, published on Sep. 23, 2010 as WO 2010/107385, which claims priority to application number 0950175-0 filed in Sweden on Mar. 20, 2009.

### FIELD OF THE INVENTION

The present invention relates to a method and system for detection of an enzyme-substrate reaction by capturing the product of the reaction on a solid support. More closely, the invention relates to a solid phase system for enzyme detection/characterisation and screening of enzyme-targeted drugs.

### BACKGROUND OF THE INVENTION

Enzymes, the catalysts of biological systems, are characterized by the constants Vₘₐₓ (the rate of reaction at maximal substrate concentration), Kₘ (Michaelis-Menten constant) and k_{cat} (maximal catalytic rate when the enzyme is fully saturated with substrate) in relation to a specific substrate. From these constants, a mechanism of action can further be determined.

Many drugs are based on enzyme inhibition. Good understanding of the mechanisms of enzyme action as well as a quick and reliable drug screening procedure are crucial for successful drug development. The majority of the existing technologies for enzymatic assays either rely on the changes of spectroscopic properties when a substrate becomes a product or are based on tedious and time-consuming radioactive assays. Alternative solutions have been suggested.

WO1990/11510A1 and WO 1990/15983A1 relates to an assay technique involving immobilising an enzyme on a solid surface and causing the enzyme to catalyse formation of a reaction product.

However, a prerequisite for the characterization of enzyme-substrate reaction is that the concentration of the substrate vastly exceeds the concentration of the enzyme and that both concentrations are well known. Moreover, the immobilized enzyme often loses its activity. For these reasons, immobilized enzymes cannot be generally used to study enzyme kinetics in the surface plasmon resonance (SPR) system.

Enzymes are frequently used as target molecules in drug development. The role of the drug in such a case is not only to bind to the target, but also to prevent the substrate to be converted into the product. The binding is not equal to the inhibition.

Alternative assays for detection of enzymatic product formation and/or inhibition are highly desirable.

### SUMMARY OF THE INVENTION

The present invention relates to a method and system for measuring enzymatic activity by detecting the product of an enzyme-substrate reaction in a flow system, comprising the following steps
a) rapidly mixing an enzyme solution with a substrate solution to start a reaction between an enzyme in said enzyme solution and a substrate in said substrate solution wherein said reaction may result in a product; and
b) monitoring any binding (i.e. the response level) of said product to a capturing agent immobilised on a solid surface and thereby obtain a Yes/No answer whether an enzymatic reaction occurs, wherein said capturing agent is directed against the product of the enzymatic reaction.

The present inventor has found that a necessary requirement for detection of an enzyme-substrate reaction is a flow system, preferably in a SPR-instrument, in which fast mixing (within seconds, preferably up to about 20 seconds, such as 5-22 seconds) between two solutions (enzyme and substrate) can be obtained (FIG. 1) with maintained good sensorgram quality.

In a preferred embodiment, the method comprises a further step:
c) measuring the initial rate of binding of the product to the capturing agent and thereby characterize the enzymatic reaction.

Preferably, the enzyme solution and substrate solution are mixed in a mixing chamber directly connected to the solid surface which is part of a flow cell. The flow cell is preferably part of a SPR (surface plasmon resonance) system.

The capturing agent immobilised on the solid surface may be any capturing agent possessing a high affinity against the product of the enzymatic reaction, preferably an antibody.

In an alternative embodiment, a compound is added before step a) and reduced/increased rate of binding in step c) compared to binding without said compound indicates that the compound inhibits/activates the enzymatic reaction on the substrate.

The compound may be a substance or fragment of a substance that activates or inhibits the enzyme reaction. The compound is preferably a drug candidate that may inhibit the enzymatic activity. Alternatively, the added compound is an enzyme activator, such as allosteric activator. Thus, besides enzyme characterization the invention provides an assay for drug screening.

In a further aspect, the invention also relates to a kit for characterising an enzymatic reaction, comprising a surface plasmon resonance sensor having a capturing reagent for the product of an enzymatic reaction bound thereto. The invention also relates to a system for characterizing an enzymatic reaction, comprising a flow cell, a mixing chamber for mixing substrate and enzyme, and a surface plasmon resonance sensor having a capture reagent for the product of an enzymatic reaction bound thereto.

### BRIEF DESCRIPTION OF THE DRAWINGS

The file of this patent contains at least one drawing/photograph executed in color. Copies of this patent with color drawing(s)/photograph(s) will be provided by the Office upon request and payment of the necessary fee.

FIG. 1 shows the basic principles of the method and system of the invention.

FIG. 2 shows sensorgrams obtained in the MBP phosphorylation experiment, in which the product was detected by the binding to an anti-phospho-MBP antibody.

### DETAILED DESCRIPTION OF THE INVENTION

to The major benefits of the system presented below are that it allows for (i) label-free enzyme characterization and (ii) selection of the best drug candidates by examining its inhibiting properties.

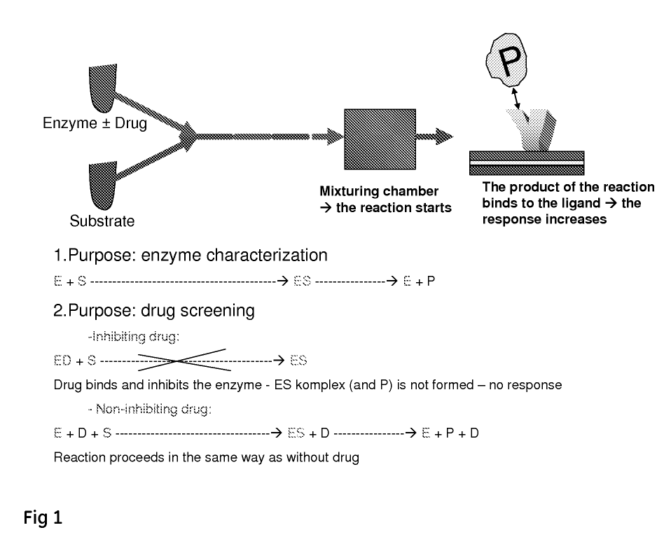

In the system (FIG. 1), an enzyme and a substrate are placed in two separate vials and subsequently injected separately (e.g. as two segments) into the flow system. These two solutions are then thoroughly and rapidly mixed using a mixing device (for example in a part of the flow system) just before reaching the immobilized capturing agent (e.g. a specific antibody) that recognizes the product of enzymatic reaction. To characterize the enzymatic reaction in terms of Vₘₐₓ, Kₘ and k_{cat}, it is important that the reaction mixture comes in contact with the capturing agent almost directly after the reaction has started to ensure that the initial rate of product formation is measured. When product binds, the response increases, which is related to the initial rate of reaction. Alternatively, the response levels can be measured to get the Yes/No answer whether the enzymatic reaction occurs.

In a drug screening approach, a drug is added to the enzyme solution. The better/more active the drug candidate, the lower the signal, compared to the reference signal given without the drug. It is possible to determine also the mechanism by which the enzyme is inhibited by the drug by performing product inhibition studies.

This kind of application requires a new technical solution to a flow system, which makes it possible to quickly and effectively mix two solutions and, more importantly, maintain good sensorgram quality.

### Examples

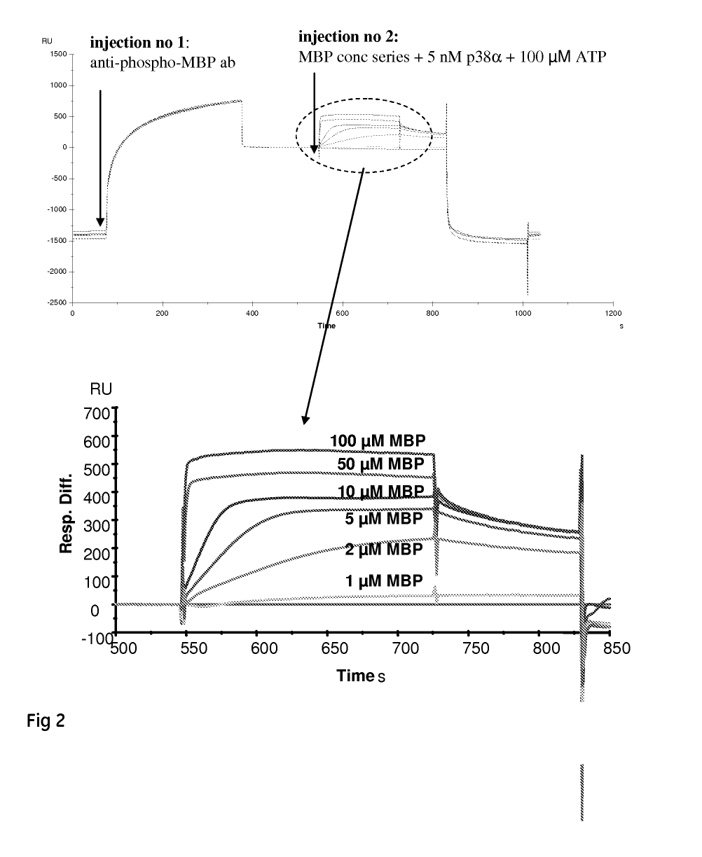

The principle of the application proposed was illustrated experimentally using the following model system:
Enzyme: p38α/(SAPK2a) kinase, Upstate, cat# 14-251, 5 nM
Substrate: MBP (Myelin basic protein), dephosphorylated, 5 mg/ml: Upstate, cat#13-110, concentration series 1-100 μM
Phosphate donor: ATP, adenosine 5′-triphosphate (Sigma), 100 μM
Capturing agent: Anti-phospho-MBP-ab, clone12, 1 mg/ml: Upstate, cat#05-429
   The instrument was Biacore T100 and chip CM5 series S. Capturing agent, mouse anti-phospho-MBP-ab, was captured on the anti-mouse Fc antibody surface prior to the injection of reaction solution.
   Reaction solutions were prepared outside the instrument. Reaction was started by adding 5 nM enzyme to the mixture of 100 μM ATP and the concentration series of dephosphorylated MBP (1-100 μM). The reaction mixture was then swiftly pipetted into the vials placed in the sample compartment, shortly before the start of injection no 2 (FIG. 2).
   MBP possesses a single Tyrosine residue that was phosphorylated using ATP as a phosphate donor and p38α kinase:

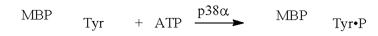

The amount of phosphorylated protein was detected by the binding of phosphortylated product to the anti-phospho-MBP antibody, which was captured on anti-Fc ab surface (FIG. 2). To determine Kₘ and Vₘₐₓ of the reaction, the slopes or report point values of binding curves (correlated to the reaction rate at given substrate concentration, v) are plotted against the substrate concentrations and the constants are determined by fitting the plot to the Michaelis-Menten equation (ref. 3):

v=Vₘₐₓ*[S]/([S]+Kₘ)

k_{cat} is given by Vₘₐₓ/[E]ₜₒₜ
The drug screening procedure, not demonstrated experimentally, relies on the qualitative analyses of the sensorgrams as described in FIG. 1. However, the skilled person would understand that the method of the invention could be used to detect inhibitors or activators of any enzymatic reaction, such as the p38α enzymatic reaction described in FIG. 2.

All patents, patent publications, and other published references mentioned herein are hereby incorporated by reference in their entireties as if each had been individually and specifically incorporated by reference herein. While preferred illustrative embodiments of the present invention are described, one skilled in the art will appreciate that the present invention can be practiced by other than the described embodiments, which are presented for purposes of illustration only and not by way of limitation. The present invention is limited only by the claims that follow.

1. A method for detecting the product from an enzyme-substrate reaction in a flow system, comprising the following steps
a. rapidly mixing an enzyme solution with a substrate solution to start an enzymatic reaction between an enzyme present in said enzyme solution and a substrate present in said substrate solution wherein said reaction may result in a product; and
b. monitoring any binding of said product to a capturing agent immobilised on a solid surface, wherein said capturing agent is directed against the product of the enzymatic reaction.

2. The method of claim 1, wherein the mixing in step a. is performed in 5 to 22 seconds.

3. The method of claim 1, further comprising:
c. measuring the initial rate of product binding to the capturing agent and thereby characterize the enzymatic reaction.

4. The method of claim 1, wherein said enzyme solution and said substrate solution are mixed in a mixing chamber directly connected to the solid surface which is part of a flow cell.

5. The method of claim 1, where the flow cell is part of a SPR (surface plasmon resonance) system.

6. The method of claim 1, wherein the capturing agent immobilised on the solid surface is an antibody or another capturing molecule directed against the product of the enzymatic reaction.

7. The method of claim 3, wherein a compound is added before step a) and reduced/increased rate of binding in step c) compared to binding without said compound indicates that the compound inhibits/activates the enzymatic action on the substrate.

8. The method of claim 7, wherein the compound is an enzyme inhibitor, such as drug candidate.

9. Method according to The method of claim 7, wherein the compound is an enzyme activator, such as an allosteric activator.

10. The method of claim 1, which is a method for drug screening.

11. A system for characterizing an enzymatic reaction, comprising:
a flow cell;
a mixing chamber for mixing substrate and enzyme; and
a surface plasmon resonance sensor having a capture reagent for the product of an enzymatic reaction bound thereto.
